(19) United States
(12) Patent Application Publication
(10) Pub. No.: US 2014/0342936 A1
(43) Pub. Date: Nov. 20, 2014

(54) METHODS AND KITS FOR DIAGNOSING LATENT TUBERCULOSIS INFECTION

(71) Applicants: INSERM (INSTITUT NATIONAL DE LA SANTE ET DE LA RECHERCHE MEDICALE), Paris (FR); UNIVERSITE MONTPELLIER 1, Montpellier Cedex 2 (FR); CENTRE HOSPITALIER UNIVERSITAIRE DE MONTPELLIER, Montpellier Cedex 5 (FR)

(72) Inventors: Pierre-Alain Rubbo, Montpellier (FR); Nicolas Nagot, Montpellier (FR); Edouard Tuaillon, Montpellier (FR); Philippe Van De Perre, Montpellier (FR)

(21) Appl. No.: 14/364,331

(22) PCT Filed: Dec. 17, 2012
(86) PCT No.: PCT/EP2012/075696

§ 371 Date: Jun. 11, 2014

(30) Foreign Application Priority Data
Dec. 15, 2011 (EP) 11306676.5

(51) Int. Cl. G01N 33/569

(52) U.S. Cl. CPC G01N 33/5695
USPC 506/9; 506/18; 506/16

(57) ABSTRACT

The present invention relates to a method for diagnosing latent tuberculosis infection in a subject comprising the step i) consisting of incubating a biological sample obtained from the subject with at least one Mycobacterium tuberculosis antigen, and thereafter a step ii) consisting of quantifying in said biological sample the secretion of at least one cytokine selected from the group consisting of IL-2, IL-15, IP-10 and MIG.

### FIELD OF THE INVENTION

The present invention relates to methods and kits for diagnosing latent tuberculosis infection.

### BACKGROUND OF THE INVENTION

A recent mathematical tuberculosis (TB) transmission modelling has shown that substantial improvements in addressing latent tuberculosis infection (LTBI) will be needed to eliminate TB before the 22^{nd} century (Hill, A. N., J. E. Becerra, and K. G. Castro. 2012. Modelling tuberculosis trends in the USA. Epidemiol Infect:1-11). Therefore, an important step towards TB elimination is the improvement of diagnostic tools for early TB infection diagnosis (2011. Early detection of tuberculosis: an overview of approaches, guidelines and tools. World Health Organization).

In most low incidence areas, Interferon (IFN)-γ release assays (IGRAs) have emerged in recent years as an accurate alternative to the tuberculin skin test (TST) for LTBI screening with higher specificity (92-99%) (Diel R, Eur Respir J 2011, Linas BP Am J Respir Crit Care Med 2011, Mazurek G H MMWR Recomm Rep 2010, Pai M Int J Tuberc Lung Dis 2009, (Pai M Ann Intern Med 2008, Menzies Ann Intern Med 2007, Sester M Eur Respir J 2011). IGRAs are based on in vitro T-cell measurements of anti-mycobacterial immunity. The T-Spot.TB test, manufactured by Oxford Immunotec, and the QuantiFERON® test, manufactured by Cellestis, are the two currently available commercial IGRAs. Although IGRAs have been considered as a major breakthrough in TB immunodetection, they lack the sensitivity normally expected from diagnostic tests in clinical practice, estimated to be 70-90% (Pai M Ann Intern Med 2008, Menzies Ann Intern Med 2007, Sester M Eur Respir J 2011). Several factors including immunodepression (such as in HIV-infected individuals) or age severely affect IGRA sensitivity results (Hang NT Plos One 2011). Accordingly, due to their sub-optimal sensitivity, the current policies on TB infection control indicate that IGRA can only be used in specific situations as a replacement for TST. For example in France, such tests are used for: i) contact investigation (age >15 years), ii) pre-employment screening of healthcare workers, iii) helping extra-pulmonary TB cases detection, and iv) initiating anti-Tumor Necrosis Factor (TNF) treatment [Test de detection de la production d′ interféron-gamma pour le diagnostic des infections tuberculeuses, Haute Autorité de Santé; 2006]. The lack of a gold standard for the diagnosis of LTBI makes it difficult to estimate the performances of IGRA or TST. Thus, most studies use newly diagnosed active TB as a surrogate for LTBI but the cell-mediated immunity being somewhat different between the two groups of patients, which possibly explains why both T-Spot.TB and QuantiFERON have sub-optimal sensitivity. The insufficient performance of IGRAs cannot be overcome by adjusting cut-offs since specificity of the tests would be affected

In HIV-positive subjects and in young children, a precise diagnosis of LTBI is crucial because such populations are at higher risk of reactivating LTBI due to their immune suppression or immature immune system, respectively. Most infected children initially have LTBI and such a population faces a high risk of progression to active TB disease (Dheda K Curr Opin Pulm Med 2009). In children up to 5 years of age, the risk of developing active TB in the 2 years post infection is 20% to 40%. The risk then decreases as age increases (Marais BJ Arch Dis Child 2007). In patients with active TB-HIV co-infection, IGRA have also poorer performances that in the general population. In particular, the sensitivity of IGRA is variable when used in population with low CD4 T cell count or in children, which lead to an increased number of false negative or indeterminate results (Raby E Plos One 2008, Bua A CMI 2011, Machingaidze S Paed Infect Dis 2011).

Therefore, to minimize false-positive and false-negative result rate, there is a need for alternative methods for the diagnosis of latent tuberculosis infection.

### SUMMARY OF THE INVENTION

The sensitivity of IGRAs is significantly improved by quantifying adequate combinations of alternative biomarkers to IFN-γ. Research by the inventors has revealed that IL-2, IL-15, IP-10, and MIG are cytokines and chemokines involved in the anti-tuberculosis defences and can be secreted in response to mycobacteria-specific stimulation. Through a lot of research, the inventors surprisingly found that quantification of these biomarkers following this stimulation could be helpful for discriminating patients with latent TB and patients without TB infection. In addition, the inventors determined that it is necessary to combine at least two proteins among IL-2, IL-15, IP-10, and MIG for improving latent TB diagnosis. Therefore they hereby propose that by combining at least two proteins having high expression in latent TB, we can obtain a joint biomarker for latent TB diagnosis. This joint marker will improve the sensitivity and specificity of latent TB detection. Moreover the method of the invention will be particularly suitable for diagnosing latent tuberculosis infection among individuals with a compromised or immature immune system.

Accordingly; the present invention relates to a method for diagnosing latent tuberculosis infection in a subject comprising the step i) consisting of incubating a biological sample obtained from the subject with at least one Mycobacterium tuberculosis antigen, and thereafter a step ii) consisting of quantifying in said biological sample the secretion level of at least two biomarkers selected from the group consisting of IL-2, IL-15, IP-10, and MIG.

### DETAILED DESCRIPTION OF THE INVENTION

The invention refers to a method allowing identification of a combination of biomarkers released after specific stimulation by mycobacteria RD1 and RD11 antigens, and their use as tools for significantly improving latent tuberculosis diagnostic, prognosis evaluation, monitoring of anti-tuberculosis treatment as well as vaccine response. The invention also refers to a kit implementing this method. The present invention overcomes the limits of IGRA.

In a first study, HCW were grouped according to QFT and tuberculin skin test (TST) results in a LTBI group (positive QFT, n=8), a LTBI-negative group (normal QFT and negative TST, n=17) and an undetermined group (sub-positive QFT and/or positive TST, n=45). Secretions of 22 cytokines were quantified using a multiparameters-based immunoassay in response to QFT-specific stimulation. As a result, thresholds discriminating LTBI from LTBI-negative HCW were established when comparing areas under the receiver operating characteristic curves for biomarkers differentially secreted between the two groups. For example, combining IL-15 and MIG, they provided a sensitivity of 100% and a specificity of 94.1% in distinguishing LTBI from LTBI-negative HCW. When using IL-15 and MIG among the undetermined group, 6/45 HCW could be classified in the LTBI group. Hence the use of additional biomarkers after IGRA screening could improve the diagnostic performance of LTBI among HCW undetermined for LTBI according to the QFT and TST results.

Accordingly, the present invention relates to a method for diagnosing latent tuberculosis infection in a subject comprising the step i) consisting of incubating a biological sample obtained from the subject with at least one Mycobacterium tuberculosis antigen, and thereafter a step ii) consisting of quantifying in said biological sample the secretion level of at least two biomarkers selected from the group consisting of IL-2, IL-15, IP-10, MIG and MIP-1β.

The subject may be any subject who was exposed to Mycobacterium tuberculosis or who was susceptible to be exposed to Mycobacterium tuberculosis. Typically, the subject is a healthcare worker, or a subject having a compromised or immature immune system, such as subjects infected with HIV. In a particular embodiment, the subject was previously screened with an IGRA assay. In some embodiments, the subject may be a non human subject. Indeed the Mycobacteria (e.g. Mycobacterium bovis) can also infect animals, especially cattle.

The term “biological sample” as used herein refers to whole blood, saliva, urine, bronchoalveolar fluids, cerebrospinal fluids, or purified peripheral blood mononuclear cells (PBMC), or any of other biological fluids, in condition that they contain leucocytes, and especially T-cells. Biological fluids has been isolated from the subject and collected in tubes or other containers containing an appropriate anti-coagulant (e.g., lithium heparin or sodium citrate). For example, the crude whole blood specimen is unfractionated whole blood collected with appropriate anti-coagulant (e.g. EDTA). It contains plasma and blood cells (red blood cells, white blood cells). It may be a freshly isolated blood sample (<48 h) or a blood sample which has been obtained previously and kept frozen until use. Typically, the biological (e.g. blood sample) comprise peripheral blood mononuclear cells (PBMCs), including T cells such as CD4 T cells, CD8 T cells, gamma-delta T cells but also monocytes, macrophages and NK cells.

In one embodiment, the incubation of the biological sample with the mycobacteria antigens is performed at the point of care locations such as physicians' offices, clinics, or outpatient facilities. Once incubation is complete, the requirement for fresh and active cells no longer exists. Cytokines are stable and, thus, the biological sample can be stored, frozen or shipped without special conditions.

Accordingly, in one embodiment, the biological sample is collected in suitable container (e.g. collection tube) containing the mycobacteria antigen or a plurality of mycobacteria antigens.

The incubation step i) may be from 5 to 48 hours, more preferably 5 to 36 hours and even more preferably 12 to 24 hours or a time period in between. Thus in one embodiment of the present invention the incubation time is 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 26 hours, 30 hours, 36 hours, 42 hours, or 48 hours.

In a particular embodiment the mycobacteria antigen is selected from the group consisting of RD1 and RD11 antigens.

As used herein, the term “RD1 antigen” and “RD11 antigen” has their general meaning in the art and refers to any antigen encoded by the region of difference 1 and 11 respectively in the genome of Mycobacterium tuberculosis. These antigens are consequently absent from all Bacille Calmette Guerin (BCG) vaccine strains and most non-tuberculous mycobacteria (exceptions include Mycobacterium kansasii, Mycobacterium marinum Mycobacterium szulgai). Typically the RD1 and RD11 antigens are selected from the group consisting of ESAT-6, CFP10, TB7.7, Ag 85, HSP-65, Ag85A, Ag85B, MPT51, MPT64, TB10.4, Mtb8.4, hspX, Mtb12, Mtb9.9, Mtb32A, PstS-1, PstS-2, PstS-3, MPT63, Mtb39, Mtb41, MPT83, 71-kDa, PPE68 and LppX. In a presently preferred embodiment the RD1 and RD11 antigens are selected from the group consisting of ESAT-6, CFP-10, and TB 7.7. Many sources for said antigens exist. Several RD1 antigens are already used in the existing commercial assays. For example, the ESAT-6 protein (early secreted antigenic target 6) is a major secreted antigen which has been purified from Mycobacterium tuberculosis short-term culture filtrates. As referred herein ESAT-6, CFP-10 (culture filtrate protein 10) and TB7.7 can be obtained from cell lysate and purification, by recombinant techniques or produced as synthetic peptides. For example ESAT-6 can be obtained as a recombinant protein from Statens Serum Institute. In another one embodiment, a plurality of RD1 and RD11 antigens are used for performing step i). Preferably, incubation of the biological sample with an amount of ESAT-6, CFP-10, and TB7.7 is preferred.

As used herein, the term “IL-2” has its general meaning in the art and refers to interleukin-2.

As used herein, the term “IL-15” has its general meaning in the art and refers to interleukin-15.

As used herein, the term “IP-10” has its general meaning in the art and refers to Interferon gamma-induced protein 10.

As used herein, the term ‘MIG” has its general meaning in the art and refers to Monokine induced by IFN-Gamma.

In a particular embodiment, the method of the present invention comprises a step ii) consisting of quantifying in said biological sample the secretion of MIG and IP-10.

In a particular embodiment, the method of the present invention comprises a step ii) consisting of quantifying in said biological sample the secretion of at least IL-15 and IP-10.

In a particular embodiment, the method of the present invention comprises a step ii) consisting of quantifying in said biological sample the secretion of at least IL-15 and MIG.

In a particular embodiment, the method of the present invention comprises a step ii) consisting of quantifying in said biological sample the secretion of at least IL-2 and IL-15.

In a particular embodiment, the method of the present invention comprises a step ii) consisting of quantifying in said biological sample the secretion of at least IL-2 and IP-10.

In a particular embodiment, the method of the present invention comprises a step ii) consisting of quantifying in said biological sample the secretion of at least IL-2 and MIG.

In a particular embodiment, step ii) comprises quantification of at least IL-15, IP-10 and MIG.

In a particular embodiment, step ii) comprises quantification of at least IL-2, IL-15, and IP-10.

Methods for quantifying secretion of a biomarker in a biological sample are well known in the art. For example, any immunological method such as but not limited to ELISA, multiplex strategies, ELISPOT, immunochromatography techniques, proteomic methods, Western blotting, FACS, or Radioimmunoassays may be applicable to the present invention.

Typically said methods comprise contacting the biological sample with a binding partner capable of selectively interacting with the biomarkers present in the biological sample. The binding partner may be an antibody that may be polyclonal or monoclonal, preferably monoclonal. In another embodiment, the binding partner may be an aptamer.

Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred.

Monoclonal antibodies of the invention or a fragment thereof can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985).

Alternatively, techniques described for the production of single chain antibodies (see e.g. U.S. Pat. No. 4,946,778) can be adapted to produce anti-cytokine, single chain antibodies. Antibodies useful in practicing the present invention also include anti-cytokine fragments including but not limited to F(ab′)₂ fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to cytokine. For example, phage display of antibodies may be used. In such a method, single-chain Fv (scFv) or Fab fragments are expressed on the surface of a suitable bacteriophage, e.g., M13. Briefly, spleen cells of a suitable host, e.g., mouse, that has been immunized with a protein are removed. The coding regions of the VL and VH chains are obtained from those cells that are producing the desired antibody against the protein. These coding regions are then fused to a terminus of a phage sequence. Once the phage is inserted into a suitable carrier, e.g., bacteria, the phage displays the antibody fragment. Phage display of antibodies may also be provided by combinatorial methods known to those skilled in the art. Antibody fragments displayed by a phage may then be used as part of an immunoassay.

In another embodiment, the binding partner may be an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. 1997. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S.D., 1999. Peptide aptamers consist of conformationally constrained antibody variable regions displayed by a platform protein, such as E. coli Thioredoxin A, that are selected from combinatorial libraries by two hybrid methods (Colas et al., 1996).

The binding partners of the invention such as antibodies or aptamers, may be labelled with a detectable molecule or substance, such as a fluorescent molecule, a radioactive molecule or any others labels known in the art. Labels are known in the art that generally provide (either directly or indirectly) a signal.

As used herein, the term “labelled”, with regard to the antibody, is intended to encompass direct labelling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labelling of the probe or antibody by reactivity with a detectable substance. An antibody or aptamer of the invention may be labelled with a radioactive molecule by any method known in the art. For example radioactive molecules include but are not limited radioactive atom for scintigraphic studies such as I123, I124, In111, Re186, Re188.

The afore mentioned assays generally involve the binding of the binding partner (ie. antibody or aptamer) to a solid support. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, plastic or glass (e.g. blood collection tubes).

In a particular embodiment, an ELISA method can be used, wherein the wells of a microtiter plate are coated with a set of antibodies which recognize said cytokine(s). A biological sample containing or suspected of containing said cytokine(s) is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

In one embodiment, an Enzyme-linked immunospot (ELISpot) method may be used. Typically, the biological sample is transferred to a plate which has been coated with the desired anti-cytokine capture antibodies. Revelation is carried out with biotinylated secondary Abs and standard colorimetric or fluorimetric detection methods such as streptavidin-alkaline phosphatase and NBT-BCIP and the spots counted.

In one embodiment, when multi-cytokine secretion quantification is required, use of beads bearing binding partners of interest may be preferred. In a particular embodiment, the bead may be a cytometric bead for use in flow cytometry. Such beads may for example correspond to BD™ Cytometric Beads commercialized by BD Biosciences (San Jose, Calif.). Typically cytometric beads may be suitable for preparing a multiplexed bead assay. A multiplexed bead assay, such as, for example, the BD™ Cytometric Bead Array, is a series of spectrally discrete beads that can be used to capture and quantify soluble antigens. Typically, beads are labelled with one or more spectrally distinct fluorescent dyes, and detection is carried out using a multiplicity of photodetectors, one for each distinct dye to be detected. A number of methods of making and using sets of distinguishable beads have been described in the literature. These include beads distinguishable by size, wherein each size bead is coated with a different target-specific antibody (see e.g. Fulwyler and McHugh, 1990, Methods in Cell Biology 33:613-629), beads with two or more fluorescent dyes at varying concentrations, wherein the beads are identified by the levels of fluorescence dyes (see e.g. European Patent No. 0 126,450), and beads distinguishably labelled with two different dyes, wherein the beads are identified by separately measuring the fluorescence intensity of each of the dyes (see e.g. U.S. Pat. Nos. 4,499,052 and 4,717,655). Both one-dimensional and two-dimensional arrays for the simultaneous analysis of multiple antigens by flow cytometry are available commercially. Examples of one-dimensional arrays of singly dyed beads distinguishable by the level of fluorescence intensity include the BD™ Cytometric Bead Array (CBA) (BD Biosciences, San Jose, Calif.) and Cyto-Plex™ Flow Cytometry microspheres (Duke Scientific, Palo Alto, Calif.). An example of a two-dimensional array of beads distinguishable by a combination of fluorescence intensity (five levels) and size (two sizes) is the QuantumPlex™ microspheres (Bangs Laboratories, Fisher, Ind.). An example of a two-dimensional array of doubly-dyed beads distinguishable by the levels of fluorescence of each of the two dyes is described in Fulton et al. (1997, Clinical Chemistry 43(9):1749-1756). The beads may be labelled with any fluorescent compound known in the art such as e.g. FITC (FL1), PE (FL2), fluorophores for use in the blue laser (e.g. PerCP, PE-Cy7, PE-Cy5, FL3 and APC or Cy5, FL4), fluorophores for use in the red, violet or UV laser (e.g. Pacific blue, pacific orange). In another particular embodiment, bead is a magnetic bead for use in magnetic separation. Magnetic beads are known to those of skill in the art. Typically, the magnetic bead is preferably made of a magnetic material selected from the group consisting of metals (e.g. ferrum, cobalt and nickel), an alloy thereof and an oxide thereof. In another particular embodiment, bead is bead that is dyed and magnetized.

In a particular embodiment, the method of the present invention further comprises a step iii) consisting of comparing the secretion level determined at step ii) with a reference value, wherein a difference between said secretion level determined at step ii) and the reference value is indicative whether said subject suffers from or does not suffer from a latent tuberculosis infection.

A reference value can be a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Preferably, the person skilled in the art may compare the cytokine secretion levels (or scores) obtained according to the method of the invention with a defined threshold value. In one embodiment of the present invention, the threshold value is derived from the cytokine secretion level (or score) determined in a control sample derived from one or more subjects who are substantially healthy (i.e. having no latent tuberculosis infection). In one embodiment of the present invention, the threshold value may also be derived from cytokine secretion level (or score) determined in a control sample derived from one or more subjects who suffers from latent tuberculosis infection. Furthermore, retrospective measurement of the cytokine secretion levels (or scores) in properly banked historical subject samples may be used in establishing these threshold values.

Typically, the optimal sensitivity and specificity (and so the threshold value) can be determined using a Receiver Operating Characteristic (ROC) curve based on experimental data. For example, after determining the levels of the cytokines in a group of reference, such as MIG and IL-15, one can use algorithmic analysis for the statistic treatment of the measured concentrations of biomarkers in biological samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator the reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In a particular embodiment the reference value are determined according to EXAMPLE 1. More particularly, the diagnostic algorithm depicted in FIG. 1 may be followed for determining the reference values.

Typically, the secretion level in a subject suffering from latent tuberculosis infection is deemed to be higher than the reference value obtained from healthy subjects.

In a particular embodiment, the method of the invention further comprises the steps pf consisting of iii) comparing the secretion levels determined at step ii) with their respective reference values, and iv) and concluding that the subject suffers from a latent tuberculosis infection when the levels determined at step ii) are higher than their respective reference values.

In another particular embodiment, a score which is a composite of the secretion levels of the different biomarkers may be also determined and compared to a reference value wherein a difference between said score and said reference value is indicative whether said subject suffers from or does not suffer from latent tuberculosis infection.

The method of the invention may be particularly suitable for monitoring the efficiency of an anti-TB treatment. Several studies have shown that IGRA tests can be useful to evaluate the anti-TB treatment in patients with active TB in low-prevalence coutries (Carrara CID 2004, Pathan J Immunol 2001, Dheda K Journal of Infection 2007, Dominguez J Diagnostic Microbiology and Infectious Disease 2009, Ribeiro BMC Infectious Diseases 2009, Latorre I Scandinavian Journal of Infect Dis 2012, Chee CBE Eur Respir J 2010). Thus, non responder patients with active TB have a persisting positive IGRA test whereas good responder to treatment have decreased IGRA results by comparison to those before initiation of treatment. To the knowledge of the inventors the usefulness of IGRA for monitoring anti-TB treatment as well as vaccine response in patients with latent TB has not been proved (Dyrhol-Riise AM BMC Infect Dis 2010, Chee CBE Am J Respir Crit. Care Med 2007, Higuchi K Respirology 2008, Pai M J Occup med Toxicol 2006, Pollock N R Infect Control Hosp Epidemiol 2009). Results indicate that LTBI patients, which are positive for IGRA tests, have regularly persistant positive IGRA tests after several months of treatment. If negativity of IGRA tests is the only condition for evaluation of treatment efficacy and driving the decision to stop treatment in LTBI subjects, therefore IGRA tests must not be used for treatment monitoring. Thus, a method quantifying combinations of biomarkers different from IFN-γ, such as proposed in our invention, can overcome this limit of the IGRA tests and improve their usefulness for monitoring efficiency of anti-TB treatment and vaccine response in patients with LTBI.

Yet another object of the invention relates to a kit for performing a method of the invention, said kit comprising means for quantifying the secretion of the biomarkers in the biological sample after step i). Typically the kit include means for determining the secretion levels in a biological sample of at least two biomarkers selected from the group consisting of IL-2, IL-15, IP-10, and MIG. The kit may also include a mycobacteria antigen or a set of mycobacteria antigens, an antibody, or a set of antibodies as above described. In a particular embodiment, the antigen, the antibody or set of antigens and antibodies are labelled as above described. The kit may also contain other suitably packaged reagents and materials needed for the particular detection protocol, including solid-phase matrices, if applicable, and standards. Since the method of the invention can be used in combination with other tests such as IGRA tests for improving latent tuberculosis diagnosis, the kit of the invention may only contain at least two different antibodies specific of each of the biomarker to identify in the sample. Thus, the kit complement the IGRA kit. In this context the method of the invention, by improving the detection of LTBI cases may be suitably used in patients for whom IGRA test results were inconclusive or indeterminate.

The invention will be further illustrated by the following figures and examples. However, these examples and figures should not be interpreted in any way as limiting the scope of the present invention.

### FIGURES

FIG. 1

The design of the diagnostic algorithm used to select additional biomarkers improving detection of latent tuberculosis (LTBI) among healthcare workers (HCW).

HCW were ranged in three groups according to QuantiFERON®-TB Gold In-Tube (QFT) and tuberculin skin test (TST). From the undetermined group, HCW were then classified into two groups according to the additional biomarker responses. IGRA, IFN-γ release assay; TB, tuberculosis.

FIG. 2

The QuantiFERON®-TB Gold In-Tube (QFT) and tuberculin skin test (TST) results of the 70 healthcare workers (HCW). QFT values were obtained after ex-vivo stimulation with ESAT-6, CFP-10 and TB7.7 antigens while TST values were the results of tuberculin stimulation. Participants were stratified into three distinct groups according to their QFT and TST results. The black lines represent the commercial cut-off value of QFT at 0.35 IU/ml and TST at 5 mm while the dotted line represents the threshold value of sub-positive QFT response (i.e. 0.1 IU/ml). IU/ml, international unit per milliliter; TB, tuberculosis.

FIG. 3

Determining additional biomarkers for the diagnosis of healthcare workers (HCW) with latent tuberculosis infection (LTBI). IL-2, IL-15, IP-10 and MIG secretion levels were quantified in the QuantiFERON®-TB Gold In-Tube (QFT) supernatants and receiver operating characteristic (ROC) curves display sensitivity versus specificity for each biomarker in differentiating the LTBI group from the LTBI-negative group. The black squares correspond to the maximum Youden's index (Y1). Areas under curves (AUC) are indicated for each panel.

FIG. 4

Identification of additional biomarkers to improve latent tuberculosis infection (LTBI) detection in healthcare workers (HCW) from the undetermined group. A) Single IL-2, IL-15, IP-10 or MIG concentrations (in pg/ml) are shown for the LTBI (black triangles), undetermined (gray circles) and LTBI-negative group of HCW (white squares) while B) two-by-two combinations of these cytokines are shown for the undetermined group of HCW only. The dotted line represents the cut-off value of each cytokines previously determined with ROC curves. The median concentrations of each biomarker for each group are shown in each panel. P<0.05 indicates a significant difference between groups using the adjusted Mann-Whitney U test. IP-10, IFN-γ induced-protein 10; MIG, Monokine induced by IFN-γ

### EXAMPLE 1

### Multi-Cytokine Detection Improves Latent Tuberculosis Diagnosis in Healthcare Workers

Summary:

Healthcare workers are at higher risk than the general population to have LTBI because they are regularly exposed to Mycobacterium tuberculosis. According to the results with the best identified combination of biomarkers (i.e. MIG and IL-15 with a threshold of 392 pg/ml and of 200 pg/ml, respectively), 100% (8/8) of LTBI cases also detected with QuantiFERON were identified, and only 6% (1/17) of the patients with a negative QuantiFERON test became positive with our method using a IL-15 and MIG combination. When this combination is applied to a third group composed of 44 patients with an “abnormal” IGRA result (i.e. negative QuantiFERON with results close to the cut-off point and/or with a positive TST), 6 patients (14%) became positive and could be considered as LTBI patients. In conclusion, among all healthcare workers tested, combination of IL-15 and MIG overcome the sub-optimal sensitivity of IGRA tests by allowing detection of 20% of LTBI cases (versus only 11% for QuantiFERON in this study). The method of the invention thus improves the diagnostic of LTBI patients.

Material & Methods

Data Collection and Participants

This study was carried out within a large multicenter study named QuantiFERON for detection of latent tuberculosis in healthcare workers (QUANTIPS), which assessed the cost-effectiveness of QFT vs. TST to detect latent tuberculosis among exposed HCW. The participants were adults from French university hospitals, working in medical units with a high risk of Mycobacterium tuberculosis exposure. In the present sub-study, the study population consisted of 70 BCG-vaccinated healthcare workers from the Respiratory Diseases and the Infectious Diseases Departments of CHRU Montpellier in France. None of the workers enrolled were infected with human immunodeficiency virus (HIV), on anti-TB treatment and/or had a clinical examination suggesting an active disease. At the baseline, a TST was done using the Mantoux technique unless a previous test was positive before enrolment. All TST results were checked between 48 to 72 hours later and were considered positive when the induration area was >5 mm. All TST ≦5 mm were arbitrary assigned a negative result equal to 0 mm. The QFT assay was performed at the baseline. The laboratory technicians and biologists were blinded for TST and previous QFT results. Inform consents were obtained from all participants. This study was registered under the identifier NCT00797836 and was approved by the Institutional Review Board of Assistance Publique—Hôpitaux de Paris.

Study Design

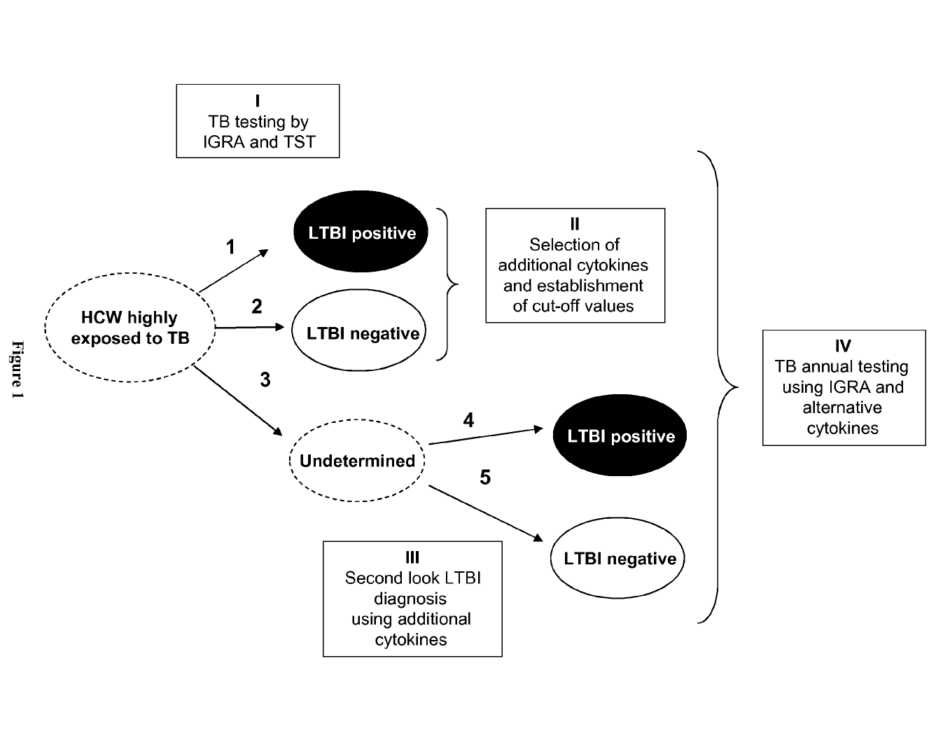

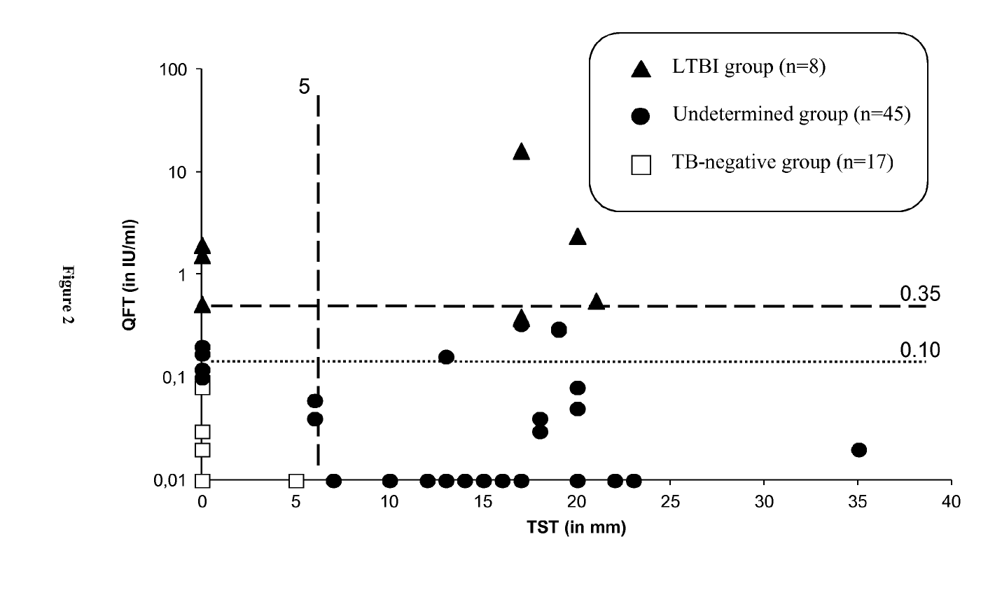

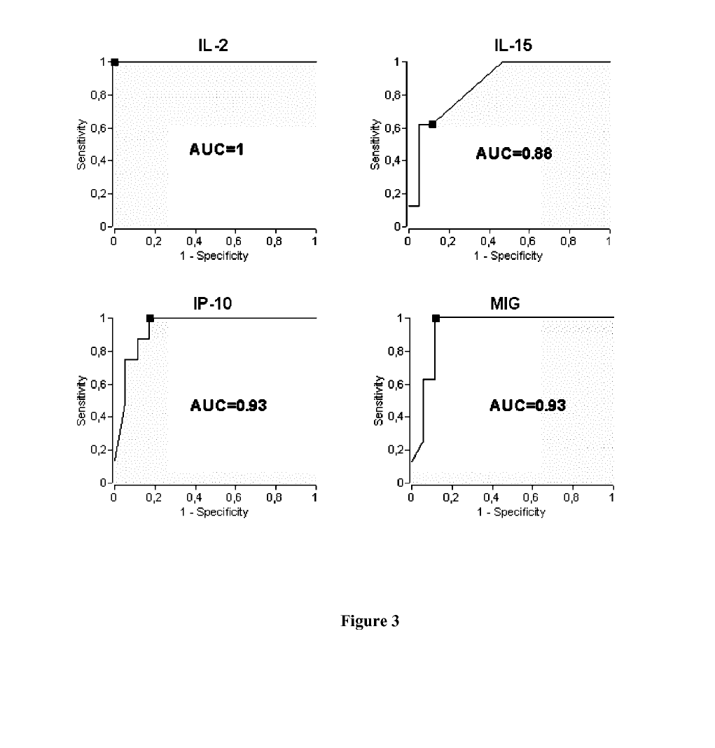

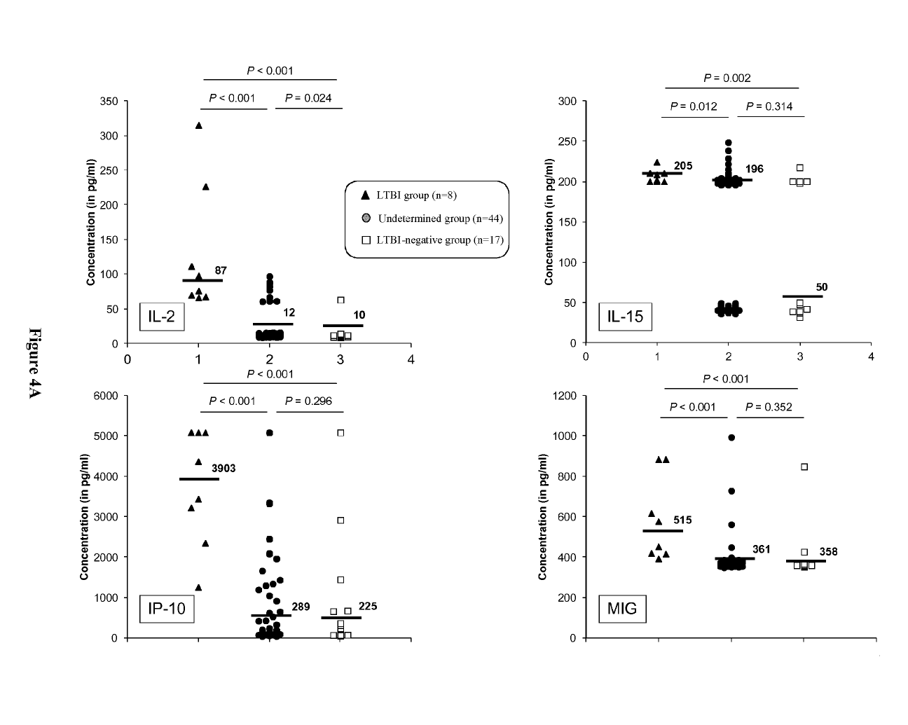

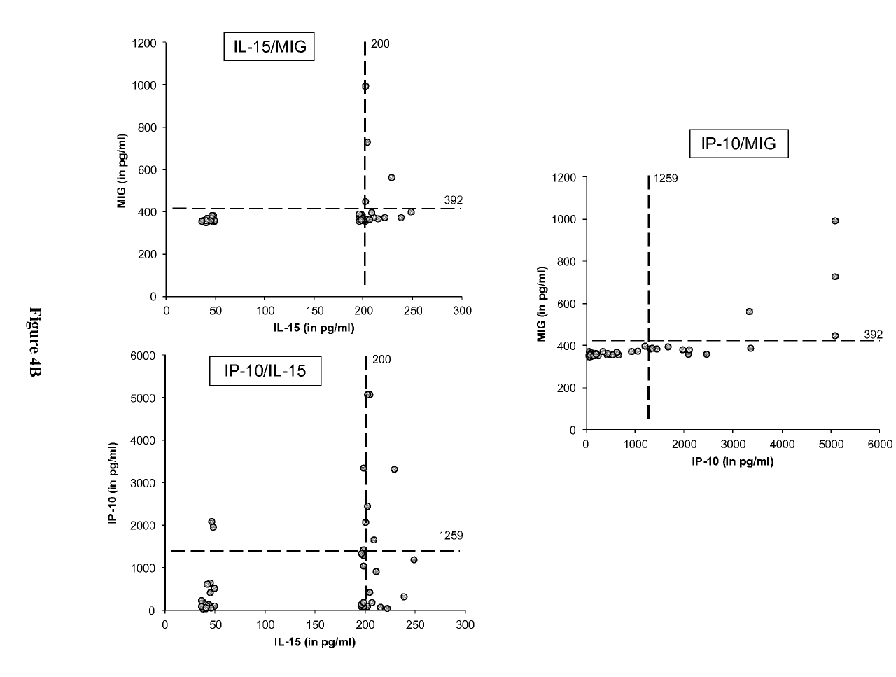

This study was conducted in two steps as described in FIG. 1. During the first step, participants were classified in three groups according to their TB screening: i) the LTBI group was composed of HCW positive for QFT using the IFN-γ cut-off defined by the manufacturer, and independently of the TST; ii) the LTBI-negative group was composed of HCW with a normal value of QFT (under 0.1 IU/ml) and a negative TST result (≦5 mm); iii) the undetermined group was composed of HCW having a sub-positive QFT result (between 0.1 and 0.35 IU/ml) and/or a positive TST (>5 mm) (FIG. 2).

Secondly, the concentration of additional cytokines secreted were measured in responses to in vitro RD1 stimulation during the QFT assays. Cytokines with a concentration that discriminated between the LTBI and the LTBI-negative group were selected using receiver operating characteristic (ROC) curves. The selected cytokines were then used with their positive cut-off values to identify HCW from the undetermined group likely to have LTBI.

The QuantiFERON®-TB Gold In-Tube Assay

The whole blood stimulation and quantification of IFN-γ production were performed according to manufacturer's instructions (Cellestis, Darmstadt, Germany). Briefly, 1 ml of venous blood was collected into each of three separate heparinized tubes: a mock stimulation for a negative control (Nil), a mitogen for a positive control and a mixture of three Mycobacterium tuberculosis-specific antigens (ESAT-6, CFP-10 and TB7.7). The tubes were incubated at 37° C. with 5% CO₂ for 24 hours and then centrifuged at 3000 g for 15 minutes. Plasma was collected and IFN-γ concentration was measured using an ELISA assay with the reagents included in the QFT kit. The optical density (OD) was read using a 450 nm filter and an ELISA plate reader. Positivity for QFT was first defined on the basis of the IFN-γ threshold recommended by the manufacturer (>0.35 IU/ml). Another QFT threshold was defined and set as the mean value+1 standard deviation (SD) of QFT results among HCW who were negative for both TST (≦5 mm) and QFT test (<0.35 IU/ml). HCW with IFN-γ levels above this new threshold (0.1 IU/ml) were considered as having a sub-positive QFT result, suggesting a possible LTBI in this population exposed to TB. Remaining plasma samples were stored at −80° C. until quantification of cytokines was carried out.

Cytokine Profile in Cell-Free Culture Supernatant of QFT

Cytokine secretion was measured in cell-free culture supernatants of HCW after 18 hours of stimulation by ESAT-6, CFP-10 and TB7.7 peptides from the QFT assay. Secretions of IL-1RA, IL-2, IL-2R, IL-4, IL-5, IL-6, IL-7, IL-10, IL-12p40/70, IL-13, IL-15, IL-17, Tumor necrosis factor (TNF)-α, Granulocyte macrophage colony-stimulating factor (GM-CSF), Macrophage inflammatory protein (MIP)-1α (CCL3), MIP-1β (CCL4), IP-10 (CXCL10), Monokine induced by IFN-α (MIG) (CXCL9), Eotaxin (CCL11), Rantes (CCL5), Monocyte chimioattractant protein (MCP)-1 (CCL2) and IFN-α were quantitated by a microbeads-based multiplex method (Cytokine human panel, Invitrogen, Villebon sur Yvette, France) and a Luminex 100 apparatus (Luminex, Oosterhout, The Netherlands) according to manufacturer's instructions. Data were acquired using 7800-15200 double discriminator gate settings and analyzed using the MLX-Booster program (BMD). Standard curves were established to determine cytokine and concentrations and a minimum of 100 microspheres per analyte were used. Concentration values above the superior point of the curve were given an arbitrary value equal to the superior point of the standard curve for each biomarker.

Statistical Analysis

The median cytokine levels (interquartile range, IQR) were compared between the LTBI and LTBI-negative group using the non-parametric Mann-Whitney U test. The individual alpha errors for multiple comparisons were corrected by adjusting the P values using the Holm technique (adjusted P<0.05 was considered significant).

Then, ROC curves of the selected biomarkers were constructed by plotting the true positive samples (sensitivity) against the false positive samples (1-specificity) for each possible cut-off point. Areas under the curve (AUC) were calculated along with their 95% confidence intervals using a non-parametric approach. Cut-offs for antigen-specific IL-2, IL-15, IP-10 and MIG were estimated at various sensitivities and specificities. To avoid false positive results, a cut-off value was retained which corresponded to the optimal operating point i.e. the maximum Youden's index (YI) defined as sensitivity+specificity−1 [12]. Then, selected cytokines with their optimal cut-off were applied to the undetermined group to detect additional LTBI.

Finally, the non-parametric Spearman's rank correlation coefficient was used to evaluate associations between secretions of selected cytokines.

Results

Study Participants

Clinical characteristics of HCW are shown in table 1. Forty-five out of the 70 participants (64%) had a TST superior to the positive threshold established at 5 mm (median=17 mm, IQR 14-20) whereas 25 participants (36%) displayed a negative TST. Eight HCW were positive for QFT using the manufacturer cut-off, 45 participants had a sub-positive QFT result between 0.1 and 0.35 IU/ml and 17 had a normal value below 0.1 IU/ml. Hence, the participants were classified into three groups based on IGRA and TST values (FIG. 1). Eight out of the 70 HCW had LTBI proved by positive QFT (12%). In contrast, 17/70 HCW (24%) had a negative testing. Thus, a majority of HCW (64%) included in this study was classed in the undetermined group because of a sub-positive IFN-γ secretion in response to RD1 stimulation and/or a positive TST (FIG. 2).

Identification of Four Additional Biomarkers Discriminating LTBI from LTBI-Negative HCW

Besides IFN-γ, 22 cytokines were analyzed after overnight T-cell specific stimulation by RD1 peptides contained in QFT tubes. Some of the biomarkers such as IL-2, IL-15, IP-10 and MIG were secreted at higher concentration in the LTBI group than in the LTBI-negative group of HCW after QFT-specific stimulation (P<0.05) (table 2).

Performances of IL-2, IL-15, IP-10 or MIG Testing in HCW Positive or Negative for LTBI

Using ROC curves, the performance of IL-2, IL-15, IP-10 and MIG testing was evaluated to discriminate LTBI from LTBI-negative HCW (FIG. 3). The most discriminating cytokines were IL-2 (a threshold at 66 pg/ml giving an AUC equal to 1, 95% CI 0.XX-1), IL-15 (a threshold at 200 pg/ml for an AUC equal to 0.88, 95% CI 0.68-0.96), IP-10 (a threshold at 1259 pg/ml for an AUC equal to 0.93, 95% CI 0.75-0.98) and MIG (a threshold at 392 pg/ml for an AUC equal to 0.93, 95% CI 0.67-0.99). These four cytokines could detect all HCW from the LTBI group. Regarding the non-TB group, 7 HCW were misclassified for IL-15, 3 for IP-10 and MIG and none for IL-2 suggesting that increasing the sensitivity up to 100% will lead to a concomitant reduction of the specificity. Finally within the LTBI group, TST had a poor sensitivity (62.5%) at thresholds of 5, 10 or 15 mm to diagnose LTBI and even worse at 18 mm (25%).

LTBI Detection Using IL-2, IL-15, IP-10 or MIG in HCW Undetermined for Tuberculosis Infection.

We investigated TB infection in HCW having sub-positive QFT results and/or positive TST response using the cytokines previously selected (FIG. 4a). We observed 15 HCW undetermined for TB were positive when assessed for IL-15, 13 for IP-10 and 6 for MIG and IL-2 testing (FIG. 4a).

There was a positive correlation between IL-15 and IP-10 secretion (r=0.57, P<0.001), IL-15 and MIG (r=0.69, P<0.001), as well as between IP-10 and MIG (r=0.78, P<0.001) (table 3). IL-2 secretion was also correlated to IL-15 (r=0.55, P<0.001), IP-10 (r=0.39, P=0.001) and MIG (r=0.49, P<0.001) (table 3).

Improving Specificity by Using Combination of Biomarkers.

IL-2 appeared as a perfect marker in our sample, with a specificity and a sensitivity of 100%. Although IL-2 is certainly a marker of interest, such a perfect diagnostic performance could be a pure luck; its true value of sensitivity is between 63% and 100%, and its true value of specificity lies between 80% to 100%. As a result, we decided to consider IL-2 by itself for further analyses. We also investigated in parallel whether an approach combining the other biomarkers could increase the specificity of the LTBI diagnostic test, which was 58.8%, 82.4% and 88.2% by using single IL-15, IP-10 or MIG (FIG. 4b). It was observed that the sensitivity was still 100% and the specificity had increased up to 94.1% when HCW had both IL-15≧200 pg/ml and MIG≧392 pg/ml. In contrast, the two-by-two combinations of MIG and IP-10 (sensitivity=100%, specificity=88.2%) or IL-15 and IP-10 (sensitivity=100%, specificity=88.2%), as well as the IL-15/IP-10/MIG association (sensitivity=100%, specificity=94.1%), did not provide better results than MIG alone in the two first cases (sensitivity=100%, specificity=88.2%) and MIG/IL-15 combination (sensitivity=100%, specificity=94.1%) in the latter case.

Using the combination of IL-15 and MIG, 6 of the undetermined HCW could have LTBI. Using IL-2, 6 HCW for the undetermined group had values above the thresholds. Among them, 4 were also positive for both IL-15 and MIG, which makes their LTBI diagnosis very likely.

Discussion:

A reliable diagnosis of LTBI in the pre-employment visit is crucial among exposed HCW to document (or exclude) an occupational contamination during subsequent medical surveillance. In 2011, QFT assay stands as the most specific immunoassay dedicated to LTBI screening. However, the IGRAs sensitivity to detect LTBI is questionable, as these tests were mainly evaluated against active TB defined by a positive microbiological test (either the microscopic detection of acid-fast bacilli in sputum or sputum culture). Recently, a 75% sensitivity and a 37% specificity have been reported in smear-negative subjects using the QFT assay, suggesting the IGRAs performance could be worse among smear-negative patients than among smear-positive subjects even in high-burden country [13]. This insufficient sensitivity of the QFT assay cannot simply be overcome by reducing the positive cut-off since it would have an impact on the specificity.

The combination of TST and QFT could be an alternative in improving the LTBI diagnostic performance [14, 15]. However, TST itself has a poor sensitivity evaluated at 80% [4] and a poor specificity among individuals vaccinated with BCG, which is the case of most HCWs exposed to TB. Our results confirm that this test is unlikely to be useful in this context unless it is used in combination with IGRA. In particular, we observed that all but one HCW belonging to the second look LTBI diagnosis group exhibits the lowest values of QFT despite a large secretion of selected cytokines such as IL-2, IL-15, IP-10 and MIG. Therefore, these HCW would have been considered as LTBI-negative subjects whether we would not have taken into account the TST results in identifying our groups in this study.

Our approach to improve the diagnosis of LTBI consisted of increasing the performance of the current IGRA based on the detection of IFN-γ only after specific stimulation. Among the QFT-negative HCW, we identified those who were very unlikely to have LTBI, on the basis of both a total absence of IFN-γ response and a fully negative TST (<5 mm). All other QFT-negative patients were classified as ‘undeterminate’, i.e. with a possible diagnosis of LTBI.

The HCW undetermined for LTBI had a QFT value above the range generally observed in healthy controls (0.01-0.1 IU/ml) suggesting that effector memory T-cells directed against RD1 antigens may be present in the exposed subjects. Using a multiplex method, we found that IL-2, IL-15, MIG and may be IP-10 detected along IFN-γ in QFT-Gold assay could be very useful biomarkers to differentiate subjects with or without LTBI. These biomarkers allowed identifying up to 15 QFT-negative individuals with LTBI (i.e. with IL-15), which is consistent with the ‘expected’ 20% false-negative tests (i.e. 14/70 people) given the 80% sensitivity of QFT. Notably, this result was obtained without substantially weakening the specificity.

Alike IFN-γ used in the commercial IGRAs, the cytokines we identified in our study in response to RD1 stimulation are involved in the T helper type 1 (Th1) cellular immune response. IL-2, MIG, IP-10 and IL-15 are pivotal in the clearance of bacteria such as Mycobacterium tuberculosis [16-19]. Our results are coherent with recent findings indicating that the measurement of T-cells directed against TB and secreting IP-10 may be useful for TB diagnosis [9-11, 20]. In addition, MIG is expressed by IFN-γ-stimulated cells in TB [21]. Functionally related to IP-10 [22, 23], MIG was detected in the bronchial epithelium, which concurs to the recruitment of activated T-cells during tuberculosis [24]. IL-15 shares many biological properties with IL-2 despite having no sequence homology [25, 26]. Unlike IP-10, MIG and IL-15 have not yet been well documented in the case of latent TB diagnosis. To our knowledge, both MIG and IL-15 have mainly been studied with the aim to discriminate active from latent TB, and only one recent report suggested that these biomarkers may be reliable cytokines to detect TB [27]. Given MIG and IP-10 secretions result from an amplification loop driven by IFN-γ, the detection of T-cell response against RD1 based on these two cytokines is probably more sensitive. Testing several parameters may limit false negative results due to inter-individual variations in the immune response during TB infection. Recent technical advances in multiparameter immunoassays using microparticles as solid supports should facilitate TB diagnosis.

In conclusion, adding IL-2, IL-15, MIG and IP-10 along with or instead of IFN-γ markedly improve the performance of IGRAs when detecting LTBI. Among HCW, a QFT assay could be first used to identify LTBI individuals using the commercial threshold. Then, the QFT supernatant of all patients with some IFN-γ response to Mycobacterium tuberculosis stimulation (i.e. value>0.1 IU/mL) should be tested for at least two biomarkers form IL-2, IL-15, MIG and IP-10 to detect additional LTBI cases.

TABLE 1
Clinical characteristics of the 70 healthcare workers
including in the study.

| Characteristics | | |
|---|---|---|
| Median Age [IQR^{a}] in years | 44 | [36-50] |
| Female gender | 59/70 | (84.3%) |
| Working groups | | |
| Doctors | 8/70 | (11%) |
| Nurses | 28/70 | (40%) |
| Auxiliary nurses | 24/70 | (34%) |
| Paramedical staff | 1/70 | (1%) |
| Other hospital workers | 9/70 | (13%) |
| Hospital departments | | |
| Tropical and Infectious | 45/70 | (64%) |
| Diseases | | |
| Pneumology | 25/70 | (36%) |
| QFT^{b} results | | |
| Negative | 62/70 | (89%) |
| Positive | 8/70 | (11%) |
| TST^{c} in mm | | |
| Negative (≦5) | 25/70 | (36%) |
| Positive (>5) [median-IQR] | 45/70 | (64%) [17-14-20] |
| Abnormal Chest X-ray | 4/70 | (6%) |

^{a}IQR, interquartile range (25^{th}-75^{th} percentiles)
^{b}QFT, quantiFERON ® TB-Gold In-Tube
^{c}TST, tuberculin skin test

TABLE 2
Concentrations of cytokines secreted by peripheral blood mononuclear
cells from healthcare workers according to QuantiFERON ®-TB
Gold In-Tube results.

| | Latent TB^{a} | | LTBI-negative | | |
|---|---|---|---|---|---|
| | HCW^{b} (n = 8) | | HCW (n = 17) | | P |
| Markers | Median (IQR^{d}) | | Median (IQR) | | values^{c} |
| IL-1RA | 8491 | (6966-9885) | 5837 | (5528-6869) | 0.140 |
| IL-2 | 87 | (69-140) | 10 | (9-12) | <0.001 |
| IL-2R | 1751 | (1696-1817) | 1714 | (1678-1751) | 0.242 |
| IL-4 | 271 | (267-282) | 263 | (261-269) | 0.126 |
| IL-5 | 15 | (14-23) | 13 | (13-15) | 0.323 |
| IL-6 | 656 | (430-800) | 546 | (335-937) | 0.852 |
| IL-7 | 133 | (133-134) | 133 | (131-133) | 0.435 |
| IL-10 | 8 | (6-15) | 7 | (6-10) | 0.448 |
| IL-12p40/ | 848 | (783-894) | 857 | (823-892) | 0.764 |
| 70 | | | | | |
| IL-13 | 31 | (24-56) | 28 | (22-31) | 0.263 |
| IL-15 | 205 | (200-211) | 50 | (39-200) | 0.021 |
| IL-17 | 208 | (206-208) | 208 | (208-208) | 0.545 |
| TNF-α | 167 | (151-195) | 161 | (152-220) | 0.815 |
| GM-CSF | 262 | (210-266) | 261 | (54-263) | 0.300 |
| MIP-1α | 2436 | (1607-2728) | 2193 | (1078-4506) | 0.966 |
| MIP-1β | 4037 | (2286-4869) | 1658 | (768-1796) | 0.061 |
| IP-10 | 3903 | (3004-5080) | 225 | (76-668) | 0.004 |
| MIG | 515 | (419-684) | 358 | (358-361) | 0.003 |
| Eotaxin | 117 | (106-130) | 106 | (89-145) | 0.618 |
| Rantes | 16760 | (15483-16760) | 13402 | (11651-16760) | 0.216 |
| MCP-1 | 15197 | (5395-22172) | 5449 | (5131-7138) | 0.107 |
| IFN-α | 109 | (108-110) | 108 | (108-109) | 0.181 |

^{a}TB, tuberculosis
^{b}HCW, healthcare workers
^{c}Mann-Whitney test comparing the two groups, with P < 0.05 statistically significant
^{d}IQR, interquartile range

TABLE 3
Spearman correlation coefficient between two-by-two combinations of IL-2,
IL-15 IP-10 and MIG secretion levels after QuantiFERON ®-TB
Gold In-Tube in 69 healthcare workers.

| Spearman | | | | |
|---|---|---|---|---|
| coefficients (P values^{a}) | IL-2 | IL-15 | IP-10^{b} | MIG^{c} |
| IL-2 | | 0.55 (<0.001) | 0.39 (0.001) | 0.49 (<0.001) |
| IL-15 | 0.55 (<0.001) | | 0.57 (<0.001) | 0.69 (<0.001) |
| IP-10 | 0.39 (0.001) | 0.57 (<0.001) | | 0.78 (<0.001) |
| MIG | 0.49 (<0.001) | 0.69 (<0.001) | 0.78 (<0.001) | |

^{a}Mann-Whitney test, with P < 0.05 statistically significant
^{b}IP-10, IFN-γ induced-protein 10
^{c}MIG, Monokine induced by IFN-γ

### REFERENCES

Throughout this example, various references describe the state of the art to which this invention pertains. The disclosures of these references are hereby incorporated by reference into the present disclosure.
1. Joshi R, Reingold A L, Menzies D, Pai M. Tuberculosis among health-care workers in low- and middle-income countries: a systematic review. PLoS Med 2006; 3: e494.
2. Menzies D, Joshi R, Pai M. Risk of tuberculosis infection and disease associated with work in health care settings. Int J Tuberc Lung Dis 2007; 11: 593-605.
3. Zwerling A, van den H of S, Scholten J, Cobelens F, Menzies D, Pai M. Interferon-gamma release assays for tuberculosis screening of healthcare workers: a systematic review. Thorax 2011.
4. Pai M, Zwerling A, Menzies D. Systematic review: T-cell-based assays for the diagnosis of latent tuberculosis infection: an update. Ann Intern Med 2008; 149: 177-184.
5. Andersen P, Munk M E, Pollock J M, Doherty T M. Specific immune-based diagnosis of tuberculosis. Lancet 2000; 356: 1099-1104.
6. Test de detection de la production d'interferon-gamma pour le diagnostic des infections tuberculeuses: Haute Autorité de Santé; 2006.
7. Diel R, Goletti D, Ferrara G, Bothamley G, Cirillo D, Kampmann B, Lange C, Losi M, Markova R, Migliori G B, Nienhaus A, Ruhwald M, Wagner D, Zellweger J P, Huitric E, Sandgren A, Manissero D. Interferon-gamma release assays for the diagnosis of latent Mycobacterium tuberculosis infection: a systematic review and meta-analysis. Eur Respir J 2011; 37: 88-99.
8. Sester M, Sotgiu G, Lange C, Giehl C, Girardi E, Migliori G B, Bossink A, Dheda K, Diel R, Dominguez J, Lipman M, Nemeth J, Ravn P, Winkler S, Huitric E, Sandgren A, Manissero D. Interferon-gamma release assays for the diagnosis of active tuberculosis: a systematic review and meta-analysis. Eur Respir J 2011; 37: 100-111.
9. Ruhwald M, Dominguez J, Latorre I, Losi M, Richeldi L, Pasticci M B, Mazzolla R, Goletti D, Butera O, Bruchfeld J, Gaines H, Gerogianni I, Tuuminen T, Ferrara G, Eugen-Olsen J, Ravn P. A multicentre evaluation of the accuracy and performance of IP-10 for the diagnosis of infection with M. tuberculosis. Tuberculosis (Edinb) 2011; 91: 260-267.
10. Ruhwald M, Petersen J, Kofoed K, Nakaoka H, Cuevas L E, Lawson L, Squire S B, Eugen-Olsen J, Ravn P. Improving T-cell assays for the diagnosis of latent TB infection: potential of a diagnostic test based on IP-10. PLoS One 2008; 3: e2858.
11. Walzl G, Ronacher K, Hanekom W, Scriba T J, Zumla A. Immunological biomarkers of tuberculosis. Nat Rev Immunol 2011; 11: 343-354.
12. Youden W J. Index for rating diagnostic tests. Cancer 1950; 3: 32-35.
13. Ling D I, Pai M, Davids V, Brunet L, Lenders L, Meldau R, Calligaro G, Allwood B, van Zyl-Smit R, Peter J, Bateman E, Dawson R, Dheda K. Are interferon-{gamma} release assays useful for diagnosing active tuberculosis in a high-burden setting? Eur Respir J 2011; 38: 649-656.
14. Fox B D, Kramer M R, Mor Z, Preiss R, Rusanov V, Fuks L, Peled N, Haim I, Raz M, Shitrit D. The QuantiFERON-TB-GOLD assay for tuberculosis screening in healthcare workers: a cost-comparison analysis. Lung 2009; 187: 413-419.
15. Pooran A, Booth H, Miller R F, Scott G, Badri M, Huggett J F, Rook G, Zumla A, Dheda K. Different screening strategies (single or dual) for the diagnosis of suspected latent tuberculosis: a cost effectiveness analysis. BMC Pulm Med 2010; 10: 7.
16. Forbes E K, Sander C, Ronan E O, McShane H, Hill A V, Beverley P C, Tchilian E Z. Multifunctional, high-level cytokine-producing Th1 cells in the lung, but not spleen, correlate with protection against Mycobacterium tuberculosis aerosol challenge in mice. J Immunol 2008; 181: 4955-4964.
17. Hasan Z, Jamil B, Ashraf M, Islam M, Dojki M, Irfan M, Hussain R. Differential live Mycobacterium tuberculosis-, M. bovis BCG-, recombinant ESAT6-, and culture filtrate protein 10-induced immunity in tuberculosis. Clin Vaccine Immunol 2009; 16: 991-998.
18. Qiu L, Huang D, Chen C Y, Wang R, Shen L, Shen Y, Hunt R, Estep J, Haynes B F, Jacobs W R, Jr., Letvin N, Du G, Chen Z W. Severe tuberculosis induces unbalanced up-regulation of gene networks and overexpression of IL-22, MIP-1alpha, CCL27, IP-10, CCR4, CCR5, CXCR3, PD1, PDL2, IL-3, IFN-beta, TIMI, and TLR2 but low antigen-specific cellular responses. J Infect Dis 2008; 198: 1514-1519.
19. Talreja J, Bhatnagar A, Jindal S K, Ganguly N K. Influence of Mycobacterium tuberculosis on differential activation of helper T-cells. Clin Exp Immunol 2003; 131: 292-298.
20. Ruhwald M, Bodmer T, Maier C, Jepsen M, Haaland M B, Eugen-Olsen J, Ravn P. Evaluating the potential of IP-10 and MCP-2 as biomarkers for the diagnosis of tuberculosis. Eur Respir J 2008; 32: 1607-1615.
21. Abramo C, Meijgaarden K E, Garcia D, Franken K L, Klein M R, Kolk A J, Oliveira S C, Ottenhoff T H, Teixeira H C. Monokine induced by interferon gamma and IFN-gamma response to a fusion protein of Mycobacterium tuberculosis ESAT-6 and CFP-10 in Brazilian tuberculosis patients. Microbes Infect 2006; 8: 45-51.
22. Bai X, Chmura K, Ovrutsky A R, Bowler R P, Scheinman R I, Oberley-Deegan R E, Liu H, Shang S, Ordway D, Chan E D. Mycobacterium tuberculosis increases IP-10 and MIG protein despite inhibition of IP-10 and MIG transcription. Tuberculosis (Edinb) 2011; 91: 26-35.
23. Sgadari C, Farber J M, Angiolillo A L, Liao F, Teruya-Feldstein J, Burd P R, Yao L, Gupta G, Kanegane C, Tosato G. Mig, the monokine induced by interferon-gamma, promotes tumor necrosis in vivo. Blood 1997; 89: 2635-2643.
24. Sauty A, Dziejman M, Taha R A, Iarossi A S, Neote K, Garcia-Zepeda E A, Hamid Q, Luster A D. The T cell-specific CXC chemokines IP-10, Mig, and 1-TAC are expressed by activated human bronchial epithelial cells. J Immunol 1999; 162: 3549-3558.
25. Waldmann H, Cobbold S. How do monoclonal antibodies induce tolerance? A role for infectious tolerance? Annu Rev Immunol 1998; 16: 619-644.
26. Waldmann T, Tagaya Y, Bamford R. Interleukin-2, interleukin-15, and their receptors. Int Rev Immunol 1998; 16: 205-226.
27. Frahm M, Goswami N D, Owzar K, Hecker E, Mosher A, Cadogan E, Nahid P, Ferrari G, Stout J E. Discriminating between latent and active tuberculosis with multiple biomarker responses. Tuberculosis (Edinb) 2011; 91: 250-256.

1. A method for diagnosing latent tuberculosis infection in a subject comprising the step i) consisting of incubating a biological sample obtained from the subject with at least one Mycobacterium tuberculosis antigen, and thereafter a step ii) consisting of quantifying in said biological sample the secretion level of at least two biomarkers selected from the group consisting of IL-2, IL-15, IP-10, and MIG.

2. The method according to claim 1 which comprises a step ii) consisting of quantifying in said biological sample the secretion of MIG and IP-10.

3. The method according to claim 1 which comprises a step ii) consisting of quantifying in said biological sample the secretion of IL-15 and IP-10.

4. The method according to claim 1 which comprises a step ii) consisting of quantifying in said biological sample the secretion of IL-15 and MIG.

5. The method according to claim 1 which comprises a step ii) consisting of quantifying in said biological sample the secretion of IL-2 and IL-15.

6. The method according to claim 1 which comprises a step ii) consisting of quantifying in said biological sample the secretion of IL-2 and IP-10.

7. The method according to claim 1 which comprises a step ii) consisting of quantifying in said biological sample the secretion of IL-2 and MIG.

8. The method according to claim 1 wherein the subject is a healthcare worker, or a subject having a compromised or immature immune system, such as subjects infected with HIV.

9. The method according to claim 8 wherein the subject for whom IGRA test results were inconclusive or indeterminate.

10. The method according to claim 1, wherein the step i) consists of incubating the biological sample with an amount of ESAT-6, CFP-10, and TB7.7.

11. The method according to claim 1, wherein the biological sample is a blood sample.

12. The method according to claim 1, which further comprises the steps of consisting of iii) comparing the secretion levels determined at step ii) with their respective reference values, and iv) and concluding that the subject suffers from a latent tuberculosis infection when the levels determined at step ii) are higher than their respective reference values.

13. A kit comprising means for determining the secretion levels in a biological sample of at least two biomarkers selected from the group consisting of IL-2, IL-15, IP-10, and MIG.

14. The kit according to claim 13 which includes a mycobacteria antigen or a set of mycobacteria antigens.

15. The kit according to claim 14 comprising at least two different antibodies specific for biomarkers selected from the group consisting of IL-2, IL-15, IP-10, and MIG.
